(19) United States
(12) Patent Application Publication
(10) Pub. No.: US 2015/0004702 A1
(43) Pub. Date: Jan. 1, 2015

(54) SYSTEM FOR ISOLATING STROMAL VASCULAR FRACTION (SVF) CELLS FROM THE ADIPOSE TISSUE AND A METHOD THEREOF

(71) Applicants: Swathi Sundar Raj, Bangalore (IN); Venkatesh Gopal, Fremont, CA (US); Nancy Priya, Bangalore (IN); Balagangadhara Krishnegowda, Bangalore (IN); Prajod Thiruvampattil, Bangalore (IN); Anish Sen Majumdar, Bangalore (IN); Murali Cherat, Bangalore (IN)

(72) Inventors: Swathi Sundar Raj, Bangalore (IN); Venkatesh Gopal, Fremont, CA (US); Nancy Priya, Bangalore (IN); Balagangadhara Krishnegowda, Bangalore (IN); Prajod Thiruvampattil, Bangalore (IN); Anish Sen Majumdar, Bangalore (IN); Murali Cherat, Bangalore (IN)

(73) Assignee: STEMPEUTICS RESEARCH PRIVATE LIMITED, Bangalore, Karnataka (IN)

(21) Appl. No.: 14/241,542

(22) PCT Filed: Aug. 28, 2012
(86) PCT No.: PCT/IB2012/054404

§ 371 Date: Feb. 27, 2014

(30) Foreign Application Priority Data
Aug. 29, 2011 (IN) 2978/CHE/2011

(51) Int. Cl. C12N 5/071; C12M 1/00; C12M 1/34

(52) U.S. Cl. CPC C12N 5/069; C12M 41/12; C12M 29/04
USPC 435/378; 435/294.1; 435/286.1

(57) ABSTRACT

The present disclosure provides an automated system for isolating stromal vascular fraction cells from the mammalian tissue. The system comprises a plurality of containers for storing buffer solutions, tissue samples and digestive buffers. A tissue processing unit fluidly connected to the containers for processing the tissues. The tissue processing unit performs at least one of washing process, digestion process, phase separation process and combination thereof for separating an aqueous fraction of tissue and a fatty fraction. A cell concentration unit fluidly connected to the tissue processing unit for receiving the aqueous fraction of tissue from the tissue processing unit. The cell concentration unit filters the aqueous fraction of tissue by vibrating a filtration assembly by a filter vibrator. A waste collection unit fluidly connectable to the tissue processing unit and cell concentration unit is provided for receiving waste tissues. The system further comprises a control unit to control the operation of the system.

### TECHNICAL FIELD

Embodiments of the present disclosure relates to a system and method for processing of biological samples, more particularly embodiments relates to the automated system and method for processing of adipose tissue to isolate stromal vascular fraction (SVF) cells.

### BACKGROUND AND PRIOR ART

Mesenchymal stem/stromal cells (MSC) can be isolated from several adult tissues such as bone marrow, adipose, placenta and umbilical cord, and are highly promising tools for regenerative medicine. While bone marrow is the most conventional source of MSC, the major limitation in its clinical application is that the concentration of MSC in bone marrow is very low. Subcutaneous adipose tissue is emerging as a promising alternative source as it has a high content of MSC, and can be easily obtained by methods such as liposuction or lipectomy.

Adipose tissue can be enzymatically disrupted to yield two main cell populations: mature adipocytes and the stromal vascular fraction (SVF). The SVF is a heterogeneous cell mixture comprising of preadipocytes, mature endothelial cells (EC), endothelial progenitor cells (EPC), vascular smooth muscle cells (SMC), pericytes, mural cells, macrophages, fibroblasts and adipose-derived stem/stromal cells (ASC). The ASC are self-renewing multipotent mesenchymal progenitors that can be easily differentiated into adipocytes, osteoblasts and chondrocytes. Additionally, several investigators have also derived endothelial, myogenic, hepatic and neuronal lineages from ASC under specific inductive conditions. In addition to their plasticity, ASC also secrete bioactive molecules such as immunomodulators and trophic, antiapoptotic, antiscarring, angiogenic, and mitotic factors. Thus, the SVF and ASC from fat tissue have enormous potential in cell-based therapy.

Non-expanded SVF cells are particularly well-suited for autologous cell therapy where clinical doses of the patient's own fat-derived stem cells can be transplanted back with minimal manipulation. SVF cells have been shown to have therapeutic benefit in several preclinical disease models, as well as in clinical trials for indications such as Crohn's disease, graft-versus-host disease, autoimmune and allergic pathologies like multiple sclerosis and inflammatory bowel disease, myocardial infarction, limb ischemia, non-healing chronic wounds, radiation injury, urinary incontinence etc. (Gimble et al. Stem Cell Research & Therapy 2010). They also have huge potential in cosmetic and reconstructive medicine as they have been shown to prolong survival of autologous fat grafts. A clinical study conducted by Yoshimura et. al. (Yoshimura et. al. Aesth Plast Surg, 2008) has demonstrated efficacy of SVF enrichment in fat grafting for breast augmentation. Fat grafting can be applied for post-surgical breast reconstruction, cosmetic breast augmentation, restructuring of facial folds, wrinkle correction and many other soft-tissue defects. Studies in animal models have shown that enrichment of fat grafts with SVF cells promotes engraftment by improving vascularization of the graft, as well as by enhancing turnover of adipocytes, and secretion of anti-apoptotic factors. In fact, the heterogenous composition of the SVF, particularly the high content of endothelial progenitor cells, is ideal for pro-angiogenic cell therapy and vascular repair. Several groups have identified CD34 positive cells in the SVF, capable of stimulating angiogenesis directly or through the release of growth factors such as IGF-1, HGF and VEGF; and SVF cells have been shown to have neo-vasculogenic potential in animal models.

Current procedures for isolation of SVF involve enzymatic digestion of the lipoaspirate tissue with collagenase, which breaks down the stromal matrix to release the SVF cells. The SVF is then separated from the fat fraction by centrifugation. The conventional procedure of isolation has several limitations in the context of clinical application:
The fat tissue needs to be transported from the hospital to a GMP-compliant laboratory.
Storage, handling and transportation of the fat tissue can affect the yield, viability and quality of cells contained in SVF.
The time taken for transportation, isolation and delivery of cells is very long.
Patient has to undergo more than one sitting at the point of care.
Cannot be used in conditions of emergency where the cells are required immediately (eg: for wound healing, burns, myocardial infarction etc.).
Bench-top open system processing requires rigorous quality control of the therapeutic product.
   A few approaches to develop an automated, closed device/system for processing stem cells are already in place. Some examples of such devices are Cytori's Celution™ system and Tissue Genesis TGI 1000™, which are presently entering clinical trials.

Ariff et al., in their published application US 20080014181, disclose an automated cell separation apparatus capable of separating cells from a tissue sample for use in cell therapies. The cell separation apparatus can be used in combination with complementary devices such as cell collection device and/or a sodding apparatus to support various therapies. The automated apparatus includes media and tissue dissociating chemical reservoirs, filters, a cell separator and a perfusion flow loop through a graft chamber which supports a graft substrate or other endovascular device. It further discloses the methods for using the tissue grafts and cell samples prepared by the devices in cell therapies.

In U.S. Pat. No. 7,514,075 and US application no. 20050084961, Hedrick et al., describe automated systems and methods for separating regenerative cells, e.g., stem and/or progenitor cells, from adipose tissue. The systems and methods disclosed herein provide rapid and reliable methods of separating and concentrating regenerative cells suitable for re-infusion into a subject.

The devices disclosed in the prior art employs centrifugal force for cell separation, which causes stress to the cells. In addition, they are expensive and bulky.

In light of foregoing discussion, it is necessary to develop a system for processing of mammalian tissues to isolate stromal vascular fraction (SVF) cells, which is economical and easy to operate in a clinical setting.

### SUMMARY OF THE DISCLOSURE

The shortcomings of the prior art are overcome and additional advantages are provided through the provision of a system and method as claimed in the present disclosure.

Additional features and advantages are realized through the techniques of the present disclosure. Other embodiments and aspects of the disclosure are described in detail herein and are considered a part of the claimed disclosure.

One embodiment of the present disclosure relates to a system for isolating cells by processing of tissue. The system comprises a plurality of containers each for storing at least one of digestive buffer, tissue sample and wash buffer solutions. A tissue processing unit is fluidly connectable with the containers for receiving the digestive buffer, tissue sample and wash buffer solutions, and processing the tissue sample. The tissue processing unit performs at least one of the washing processes, digestion process, phase separation process and a combination thereof for separating an aqueous fraction of tissue and a fatty fraction of the digested tissue sample. A cell concentration unit is fluidly connectable with the tissue processing unit for concentrating the aqueous fraction of the digested tissue. The cell concentration unit comprises; a filtration assembly including a plurality of filter chambers for sieving or size based filtration of the cells from the aqueous fraction of the digested tissue to collect cells, and a filter vibrator is attached to the filtration assembly for vibrating the filter chambers. A waste collection unit is fluidly connectable to the tissue processing unit and the filtration unit for receiving at least one of aqueous fraction and fatty fraction of the digested tissues from the tissue processing unit and the filtration unit. The system further comprises a control unit interfaced with the tissue processing unit and the filter vibrator, for controlling the operation of the tissue processing unit and the filter vibrator to obtain the cells from tissue.

In an embodiment of the present disclosure, the tissue is a mammalian tissue selected from a group comprising but not limited to adipose tissue, placental tissue and umbilical cord tissue, wherein the adipose tissue is processed for isolating the Stromal Vascular Fraction (SVF) cells and multipotent stem/stromal cells from placental and umbilical cord tissue.

In an embodiment of the present disclosure, the system comprises a plurality of peristaltic pumps and valves connectable to the containers, the tissue processing unit, the cell concentration unit and the waste collection unit for controlling flow rate of tissue samples, wash buffer solution, digestive buffer solution.

In an embodiment of the present disclosure, the containers, the tissue processing unit, the cell concentration unit, and the waste collection unit are connected to each other through a tubing system.

In an embodiment of the present disclosure, the system is preferably enclosed in a chamber, and at least one temperature sensor is placed in a chamber to measure and regulate the temperature of the chamber. The temperature sensor is interfaced with the control unit to maintain the temperature of the chamber within a predetermined limit.

In an embodiment of the present disclosure, the control unit is provided with a user interface having display and input buttons to feed in the required parameters for processing the tissue.

In an embodiment of the present disclosure, the system comprises optionally a filter waste chamber connected to a ultimate filter chamber of the filtration assembly for collecting the remaining aqueous fraction of tissues after filtration.

In an embodiment of the present disclosure, input nozzle is provided at top most filter chamber of the filtration assembly for receiving the aqueous fraction of digested tissues from the tissue processing unit, and optionally an output nozzle is provided in each filter chamber of the filtration assembly for collecting the cells of interest from each chamber when the filter chambers are mounted one above the other.

In an embodiment of the present disclosure, at least one input nozzle and at least one output nozzle is provided in each filter chamber of the filtration unit, wherein the input nozzle provided in first filter chamber configured to receive the aqueous fraction of digested tissues from the tissue processing unit and the output nozzle provided in each filter chamber is configured for supplying the sieved aqueous fraction to subsequent chamber respectively or to optionally collect the SVF cells when the filter chambers are mounted adjacent to each other.

In an embodiment of the present disclosure, at least one breather nozzle is provided in each of the filter chambers to facilitate free air flow inside the chambers. To maintain aseptic environment the breather nozzles are protected by a filter of perforations of the size ranging from 0.8 μm to 0.1 μm, preferably 0.22 μm. Breather filter material includes but not limited to PES (polyethersulfone), cellulose acetate, Teflon (PTFE) or any other known in the art.

In an embodiment of the present disclosure, the filter chambers and the filter waste chamber are mounted one above the other.

In an embodiment of the present disclosure, the filter chambers and the filter waste chamber are mounted adjacent to each other, and each of the filter chambers and the filter waste chamber are connected using a tubing system.

In an embodiment of the present disclosure, each filter chamber is provided with at least one filter cartridge. The filter cartridge comprises a filter element having predetermined perforations disposed in a housing, wherein the housing optionally comprises a plate at its bottom. The size of perforations of the of the filter element ranges from about 1 μm to about 200 μm.

Another embodiment of the present disclosure relates to a cell concentration unit, comprising of a filtration assembly including a plurality of filter chambers of predetermined shape and predetermined size, optionally a filter waste chamber is connected to one of the filter chamber. At least one filter cartridge is located in each of the filter chambers and the filter cartridge comprises a filter element having predetermined perforations disposed in a housing, wherein the housing optionally comprises a support member. A filter vibrator is placed below the filter chambers for generating vibrations for filtration.

In an embodiment of the present disclosure, the filter vibrator comprises of a rigid plate of predetermined shape configured to form a base of the filter vibrator. A plurality of guide shafts are fixed at predetermined locations on the rigid plate, each of the guide shaft comprises of a top stopper at the free end of the guide shaft and a bottom stopper at predetermined distance below the top stopper, wherein the guide shafts are arranged to pass through a movable plate. The movable plate of predetermined shape is slidably mounted on the bottom stopper of the guide shaft, wherein the movable plate is connectable to the filtration unit. At least one compression spring is mounted between the top stopper of the guide shaft and the movable plate. A cam follower is fixed to bottom end of the movable plate, the cam follower is configured to follow an amplitude generator. A motor is mounted on the rigid plate, the motor is coupled to the amplitude generator for actuating the cam follower to generate vibrations for filtration.

In an embodiment of the present disclosure, the filter vibrator comprises a pair of load bearings mounted on rigid plate, and are coupled to the amplitude generator.

Another embodiment of the present disclosure relates to a method of obtaining cells from tissues, preferably stromal vascular fraction (SVF) cells from adipose tissue using the system explained above. The method comprises acts of transferring, predetermined quantity of a tissue sample and a wash buffer solution from the containers into a tissue processing unit. Then washing the tissue samples with wash buffer solution by agitating the mixture in the tissue processing unit and allowing phase separation of the mixture to obtain a primary fatty upper fraction and a primary aqueous lower fraction in the tissue processing unit. The primary lower aqueous fraction obtained from the previous step is disposed to a waste collection unit. Then, pumping predetermined quantity of a digestive buffer from container to the tissue processing unit, and digesting the fatty upper fraction with the digestive buffer by agitating the mixture in the tissue processing unit for a predetermined time, the digestion process is optionally arrested at the end of the predetermined time period by pumping in predetermined quantity of serum or enzyme inhibitor or a combination thereof, and mixing by agitation. Alternatively the digestion process can be arrested by multiple washes. Now, allowing phase separation of the mixture in the tissue processing unit to obtain a secondary fatty upper fraction and a secondary aqueous lower fraction. Then, the secondary aqueous lower fraction is directed to a cell concentration unit. The secondary aqueous fraction is supplied to the cell concentration unit for concentrating the cells—using-a vibration assisted filtration assembly of the cell concentration unit, optionally along with removal of red blood cells to obtain said SVF cells.

In an embodiment of the present disclosure, the washing process, phase separation process and disposal of primary lower aqueous fraction process are carried out at least one time, preferably 3-4 times and the time period for the aforementioned processes ranges from about 5 minutes to about 20 minutes, preferably about 10 minutes.

In an embodiment of the present disclosure, the digestion process is carried out for time period ranging from about 15 minutes to about 2 hours, preferably from about 30 minutes to about one hour.

In an embodiment of the present disclosure, the phase separation occurs in time period ranging from about 1 minute to about 10 minutes, preferably from about 2 minutes to about 5 minutes.

In an embodiment of the present disclosure, the digestive buffer is a mixture of wash buffer and enzyme, wherein the enzyme is selected from group comprising collagenase, pepsin, trypsin, dispase and other known in art for the purpose or any combination thereof.

In an embodiment of the present disclosure, the wash buffer or buffer is selected from a group comprising normal saline, ringer's solution, lactated ringer's solution, Hank's balanced salt solution (HBSS) and any combination thereof.

In an embodiment of the present disclosure, second aqueous fraction comprises a mixture of SVF cells, undigested tissue waste and blood cells such as RBC, lymphocytes and monocytes or any combination thereof.

In an embodiment of the present disclosure, washing with the wash buffer and the digestion is carried at temperature ranging from about 35° C. to about 38° C. preferably from about 36.5° C. to about 37.5° C.

In an embodiment of the present disclosure, the optional removal of red blood cells is carried out by at least one of filtration or affinity matrix or a combination thereof.

In an embodiment of the present disclosure, the obtaining of the SVF is automated and maintains sterility throughout the process.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE ACCOMPANYING FIGURES

The novel features and characteristic of the disclosure are set forth in the appended claims. The disclosure itself, however, as well as a preferred mode of use, further objectives and advantages thereof, will best be understood by reference to the following detailed description of an illustrative embodiment when read in conjunction with the accompanying figures. One or more embodiments are now described, by way of example only, with reference to the accompanying figures wherein like reference numerals represent like elements and in which:

FIG. 1 illustrates a perspective view of an automated system for isolating stromal vascular fraction (SVF) cells from mammalian tissue of the present disclosure.

FIG. 2 illustrates a front view of an automated system for isolating stromal vascular fraction (SVF) cells from mammalian tissue of the present disclosure.

FIG. 3 illustrates a line diagram of a system for isolating stromal vascular fraction (SVF) cells from mammalian tissue of the present disclosure.

FIG. 4 illustrates block diagram of a system for isolating stromal vascular fraction (SVF) cells from mammalian tissue of the present disclosure.

FIG. 5 illustrates perspective view and magnified view of a filtration assembly of the system for isolating stromal vascular fraction (SVF) cells from mammalian tissue of the present disclosure.

FIG. 6 illustrates perspective view of the filter vibrator of the system for isolating stromal vascular fraction (SVF) cells from mammalian tissue of the present disclosure.

FIG. 7 illustrates perspective view of the cell concentration unit of the system for isolating stromal vascular fraction (SVF) cells from mammalian tissue as one embodiment of the present disclosure.

FIG. 8 illustrates perspective view of the cell concentration unit of the system for isolating stromal vascular fraction (SVF) cells from mammalian tissue as another embodiment of the present disclosure.

FIG. 9 illustrates perspective view and side view of the filtration assembly with plurality of cam followers mounted below the filtration assembly as another embodiment.

The figures depict embodiments of the disclosure for purposes of illustration only. One skilled in the art will readily recognize from the following description that alternative embodiments of the structures and methods illustrated herein may be employed without departing from the principles of the disclosure described herein.

### DETAILED DESCRIPTION OF THE INVENTION

The foregoing has broadly outlined the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that the conception and specific embodiment disclosed may be readily utilized as a basis for modifying or designing other structures for carrying out the same purposes of the present disclosure. It should also be realized by those skilled in the art that such equivalent constructions do not depart from the spirit and scope of the disclosure as set forth in the appended claims. The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

To overcome the drawbacks mentioned in the background, it is necessary to develop a point-of-care system for isolation of clinical grade SVF cells from lipoaspirated tissue. Accordingly, the present disclosure discloses an automated bench-top/table-top or portable point-of-care system for processing adipose tissue to isolate SVF, and is programmed to be operated by a user guided human interface.

It is the objective of the present disclosure is to provide a compact, closed, automated, point-of-care system; and methods for separating and concentrating clinical grade multipotent stromal/stem cells from mammalian tissue such as adipose tissue, placental tissue and umbilical cord tissue, and other biological tissues.

It is yet another objective of the present disclosure is to provide a system to process biological samples by washing and digesting the tissue sample; and further subjecting the sample to filtration in cell concentration unit using filtration assembly comprising of more than one filter chamber. The filtration can be performed by techniques such as but not limiting to simple filtration, pressure assisted filtration, vacuum assisted filtration, and vibration assisted filtration or any combination thereof. In a preferred embodiment, the filtration technique followed is vibration assisted filtration, in which the design and construction of the filter assembly incorporates a vibratory mechanism to dislodge cells and waste that clog the filters. Either by itself, or in combination, all these mechanisms improves flow rate and prevent clogging of filter materials and enable efficiency of cell concentration unit.

It is an objective of the present disclosure is to provide a system without employing centrifugal force for obtaining SVF from adipose tissue; and means to optionally remove red blood cells.

It is further an objective of the present disclosure is to provide a system for separating and concentrating SVF from adipose tissue comprising one or more—containers for tissue sample, digestive buffer and wash buffers, a tissue processing unit for washing and digestion, a cell concentration unit comprising of filtration assembly with one or more filter chambers, a waste collection unit, and means for collection of the SVF from the system.

It is still another objective of the present disclosure to provide an aseptic system having disposable and non-disposable elements. The disposable elements for one-time use prevent contamination while processing or handling of the sample. While the non-disposable elements such as electrical and electronic elements of the device are separated and housed away from the cell processing area.

In brief, the present disclosure discloses a closed, automated, point-of-care, bench-top system for isolation and processing of clinical grade stromal vascular fraction (SVF) from adipose tissue sample and a method for isolation and processing of stromal vascular fraction (SVF) from adipose tissue sample by employing the system. A point of care system would ensure that the processing, and delivery of the final cell product consumes minimal time, and the cells are delivered to the patient in a single sitting, within a couple of hours of the fat aspiration procedure in a clinical setting. The system is further provided with means to optionally remove red blood cells. The automation of the procedure eliminates the need for specialized personnel, and maintains consistency of the end product. The entire isolation procedure would be carried out in a closed automated system with clinical grade sterile disposable components and tubing elements.

Present disclosure provides an automated, closed system, point-of-care device, in order to simplify the process of isolating, concentrating and enriching stem cells from adipose tissue or any other tissue. It can process tissue samples obtained from human and other mammals for clinical and veterinary applications. This device can be used for processing of mammalian tissues to obtain clinical grade mutipotent stromal/stem cells. The mammalian tissues can be selected from group comprising adipose tissue, placental tissue, bone marrow and umbilical cord tissue or any combination thereof. This system can be used in research laboratory for research application.

The system is designed for convenient use in clinical settings/hospitals. In one of the embodiments the system is compactly designed to be used as a bench-top device; and in another embodiment, the whole system is mounted on rollers/wheels for mobility of the whole device and thus can be conveniently taken to the required location of operation where the tissue harvest procedure is conducted.

This automated system broadly comprises of two modules; Module 1 is the tissue processing unit—wherein the tissue sample is washed and is subjected to enzymatic digestion. The tissue processing unit is selected from a group comprising but not limited to Multi Planar Mixer System (MPMS), conventional mixers including electromagnetic mixer, motor driven mixers. Whereas the Module 2 is a cell concentration unit for obtaining concentrated cells preferably SVF through filtration. The filtration can be performed by techniques such as but not limiting to simple filtration, pressure assisted filtration, vacuum assisted filtration, and vibration assisted filtration or any combination thereof.

In one of the embodiments of the present disclosure, the stromal vascular fraction (SVF) processing system is an automated apparatus having all the necessary electronic components and computerized control system for mammalian tissue digestion, heating, wash, separation and concentration of cells in aseptic conditions in a clinical setting. In a preferred embodiment, the apparatus comprises of two modules: one module for digestion and washing of the collected adipose tissue sample, and it is provided with one or multiple inlets for injecting the tissue samples, wash buffers and digestive buffers; while the second module is for concentration of the cells, also provided with inlets and outlets. The final processed cells for the desired clinical application is collected from the outlet using a suitable cell collector known in the art or specially designed for such purpose. The components of the apparatus such as the tissue processing unit, buffer unit, cell concentration unit, waste collection unit, along with the tubing system connecting all the units is for single use, thus preventing contamination and infection.

In another embodiment, the SVF processing system houses all of the electronic components necessary for operation/monitoring and diagnostics of the system, along with computerized programs and software for controlling and facilitating the user interface for operation of the device. In another embodiment the device also has a communication interface for remote operation and diagnostics.

In one of the preferred embodiments, the user interface comprises of a display screen with input buttons for adjusting the required set-up for operation of the device. The user interface can be a touch screen display.

In yet another embodiment of the present disclosure the entire device is provided with a permanent non-disposable housing which provides the main framework for connecting the disposable components to assemble the device. This framework is provided with connecting means through which the tubing can be connected so as to assemble the sterile containers, washing/digestion unit, cell concentration and waste collection unit.

Operation of the device is automated through elaborate engineering of the modules and control mechanisms. The user input keypad acts as an interface for the operator to input various parameters like volume of sample, position of the valves to be activated and the sequence of activation. In one of the preferred embodiment, all parameters are pre-calculated for a given volume of tissue to be processed and displayed on the display screen. However, depending on the sample quality and application, the operator can over-ride the auto-program to create a new program for a given process.

Now, the system and the method used for isolating SVF from adipose tissues are explained with the help of figures. The figures are adopted for the purpose of illustration only and should not be construed as limitations on the arrangement.

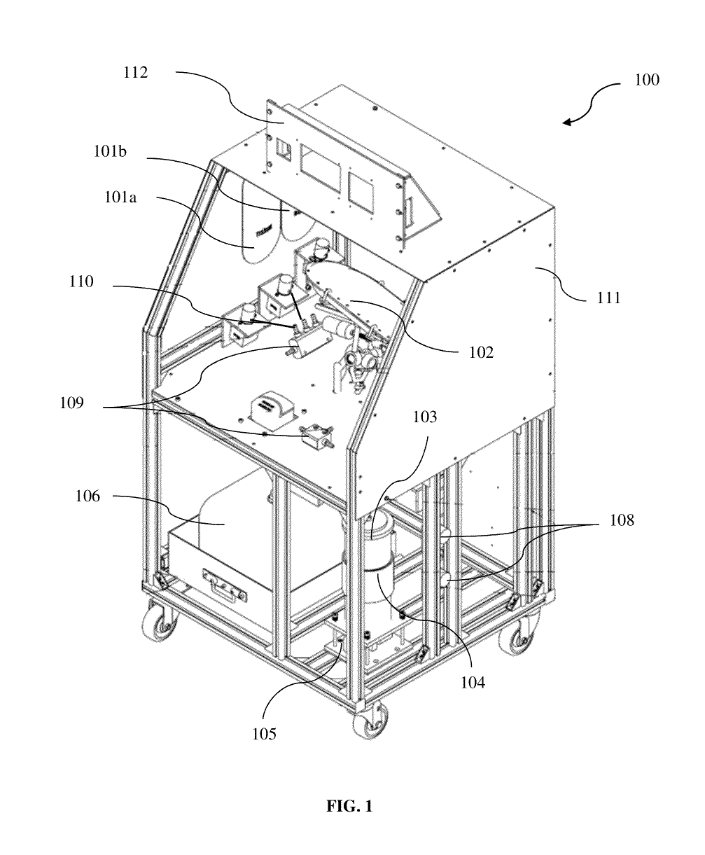

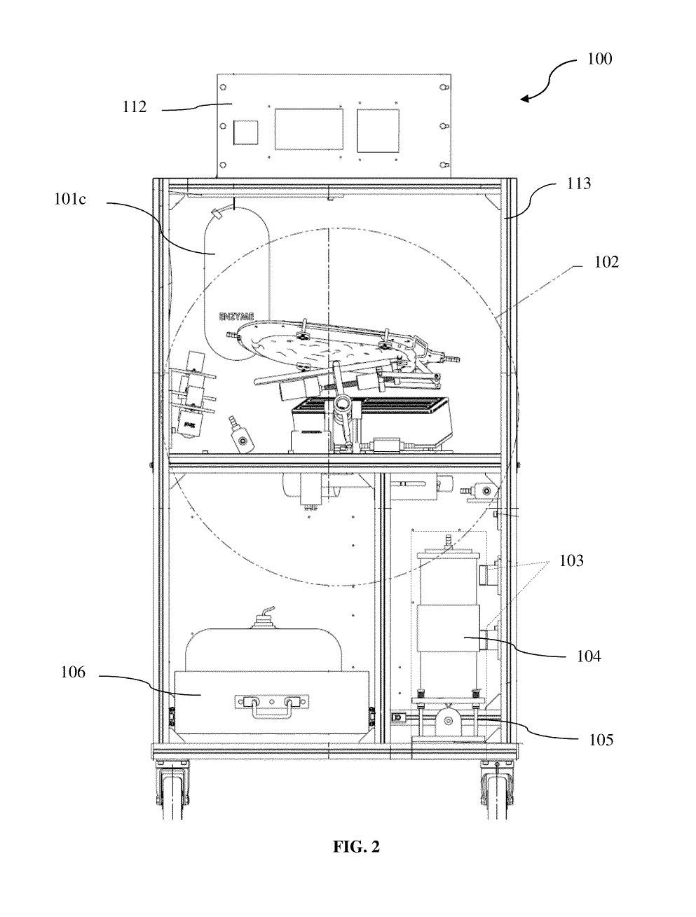

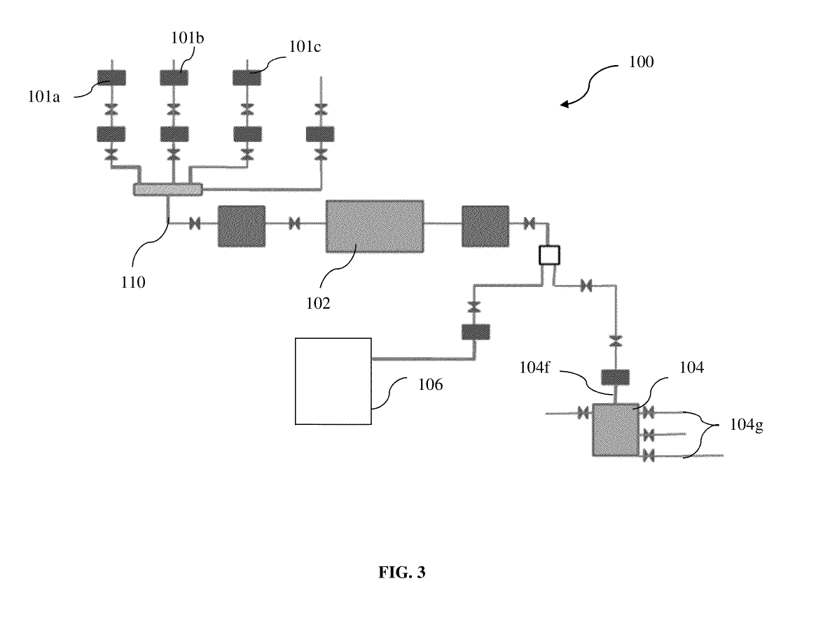

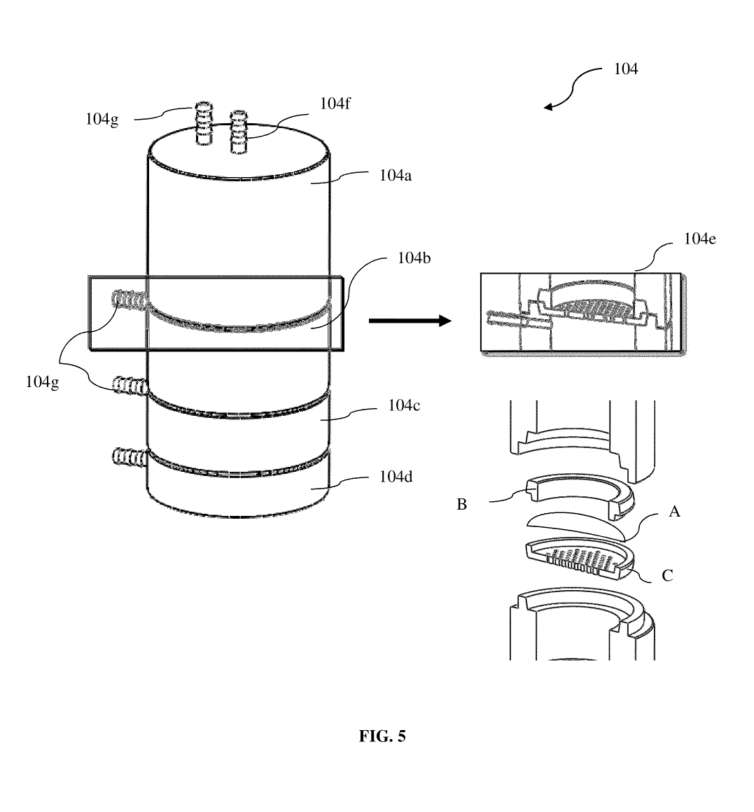

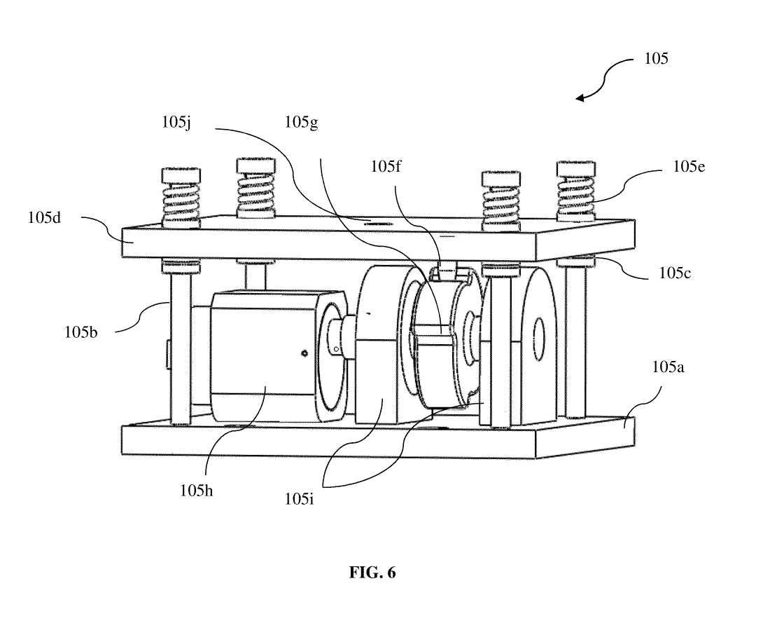

FIGS. 1 and 2 are exemplary embodiments of the present disclosure illustrating perspective view and front view of an automated system (100) for isolating stromal vascular fraction (SVF) cells from adipose tissues. The system (100) for isolating stromal vascular fraction (SVF) cells from adipose tissues samples comprises plurality of containers (101a-101c) of predetermined shape for storing tissue samples, wash buffer solution, and digestion buffer solutions. A tissue processing unit (102) also termed as washing/digestion unit fluidly connected to the containers (101a-101c) through a tubing system (110) for processing the tissue samples. The tissue processing unit (102) performs the washing process, digestion process, and phase separation process and its combination thereof for processing the tissue to separate aqueous fraction and the fatty fraction of the digested tissue. A cell concentration unit (103) is fluidly connectable to the tissue processing unit (102) through the tubing system (110) for filtering the aqueous fraction of tissue. The cell concentration unit (103) comprises, a filtration unit/assembly (104) including plurality of filter chambers (104a-104c) of predetermined shape fluidly connected to each other. And a filtration assistance mechanism connected to the filtration assembly (104). The filtration assembly (104) is also optionally provided with a filter waste chamber (104d) attached to the filter chambers (104a-104c) for collecting remaining portion of aqueous fraction tissues after the filtration. Further, each filter chamber (104a-104c) is provided with at least one filter cartridge (104e) [shown in FIG. 5] is placed between each filter chambers (104a-104c) and the filter waste chamber (104d). The filter cartridge (104e) comprises a filter element (A) with predetermined size disposed in a housing (B). The housing optionally comprises a support member (C). The filter cartridge (104e) filters the aqueous fraction of tissue received from the tissue processing unit (102) to obtain cells of interest from one of the filter chambers (104a-104c) and to collect the waste tissues in the filter waste collection unit (104d). In an embodiment of the present disclosure, the filter assistance mechanism is selected from group comprising but not limited to simple filtration, pressure assisted filtration, vacuum assisted filtration, and vibration assisted filtration or any combination thereof. In a preferred embodiment, the filtration technique followed is vibration assisted filtration The design and construction of the filter assembly incorporates a vibratory mechanism [shown in FIG. 6] to dislodge cells and waste that clog the filters. Either by itself, or in combination, all these mechanisms improve flow rate and prevent clogging of the filter elements and enable cell concentration by filtration. The system (100) further comprises a waste collection unit (106) of predetermined shape fluidly connected to the tissue processing unit (102) and the filtration assembly (104) using tubing system (110). The waste collection unit (106) is configured to collect the aqueous fraction of tissues from the tissue processing unit (102) after the washing process, fatty fraction of tissues from the tissue processing unit (102) after the digestion process and the remaining portion of aqueous fraction of tissues from the filtration assembly (104) after the process of filtration. Further, the system (100) comprises a control unit (107) [shown in FIG. 3] interfaced with the tissue processing unit (102) and the filtration assistance mechanism/filter vibrator (105) for controlling the operation of the tissue processing unit (102) and the filter vibrator (105) to obtain the Stromal Vascular Fraction (SVF) cells from the adipose tissue.

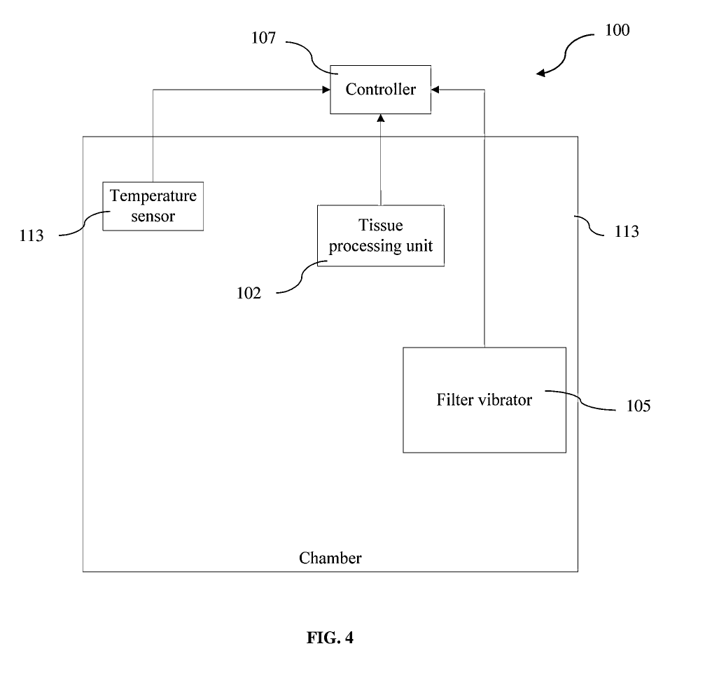

In an embodiment of the present disclosure, the system (100) comprises a plurality of peristaltic pumps (108) and a plurality of pinch valves [shown in FIG. 4] are connected between the containers (101a-101c), the tissue processing unit (102), the cell concentration unit (103) and the waste collection unit (106) using the tubing system (106). The peristaltic pumps (108) and the pinch valves are interfaced with the controller (107) to facilitate controlled flow of tissue samples, wash buffer solutions, digestive buffers, and waste fluids.

In an embodiment of the present disclosure, the tubing system (110) is made of flexible non-reactive plastic material, but not limited to silicone or Tygon with a diameter of about 0.5-5 cm range. The container (101a-101c), the tissue processing unit (102), the cell concentration unit (103) and the waste collection unit (106) are made of materials selected from group comprising but not limited to polypropylene or polystyrene. The geometry of the tissue processing (102) unit is designed such that, it provides the maximum surface area, and promotes efficient high rate of heat transfer. The shape of the tissue processing unit (102) is selected from at least one of Cylindrical, rectangular, Cylindrical with baffles/fin, flat rectangular geometry with or without using multiple stacks, honeycomb and other known suitable geometry in the art.

The tissue processing unit (102) has a working capacity of maximum 2000 ml of liquid for processing. In another embodiment, the tissue processing unit (102) is designed to process volume less than 1000 ml capacity. In yet another embodiment, the volume capacity of the tissue processing unit (102) is designed as per the requirement of volume of tissue to be processed as shown in the table 1. The volumes shown in the table 1 is for an illustration purpose and should not be construed as limitation.

TABLE 1

| Volume of tissue in (ML) | Volume of the container in (ML) |
|---|---|
| 500 | 1200 |
| 400 | 970 |
| 300 | 730 |
| 200 | 520 |
| 100 | 300 |

In another embodiment, the device is provided with separate containers for large scale and small scale processing. As per the present disclosure, large scale processing means fat sample in the range of 300-1000 ml and small scale ranges from 50-300 ml.

In an embodiment of the present disclosure, the volume capacity of tissue container (101a), buffer container (101b) and Digestive buffer container (101c) is designed as per the requirement of sample to be processed. In an embodiment, the capacity of the said containers (101a-101c) ranges from 300 ml to 10000 ml. In a preferred embodiment, the volume capacity is 5000 ml. The volume capacity of the containers (101a-101c) can be varied on a requirement basis.

The disposable elements in the system (100) comprise of containers (101a-101c), tissue processing unit (102), cell concentration unit (103), waste collection unit (106), tubing system (110) and connectors. All the disposable components used in the system (100) are of medical grade material suitable for processing biological samples meant for clinical use. All the disposable elements are sterilized by 7-irradiation or any other means known in the art, and are intended for single/one time use only, and supplied with the system (100) as a sterile package. In another embodiment the sterile packs will be interlocked with the device using RFID tags. Tubing is rated to withstand a minimum of 20 psi of pressure or greater.

The system (100) as explained above can be optionally enclosed in a chamber (111). The chamber (111) can be transparent or opaque and is configured to support all the components including containers (101a-101c), tissue processing unit (102), cell concentration unit (103), waste collection unit (106), peristaltic pumps (108) and the tubing system (110) of the system. In an embodiment of the disclosure, the geometry of the chamber can vary but not limited to cubical, square, rectangular, cylindrical and other known geometry which can be used for the purpose. In one embodiment of the present disclosure, the controller (107) is mounted on top surface of the chamber (111) and the controller is provided with a user interface having a display (112) and input buttons to feed in required parameters for processing the tissue. The display (112) can be selected from a group comprising but not limited to LCD (Liquid crystal display) display, Light emitting diode (LED) display, Cathode ray tube (CRT) display, and thin film transistor liquid crystal (TFT-LCD) display, or thin film transistor (TFT) display.

In an embodiment of the present disclosure, the system (100) comprises at least one temperature sensor (113) [shown in FIG. 4], placed in a chamber (111) to measure and regulate the temperature of the chamber (111). The temperature sensor (113) is interfaced with the control unit (107) to maintain the temperature of the chamber (111) within a predetermined limit. The temperature of the chamber (111) is maintained in range from about 35° C. to about 38° C. preferably from about 36.5° C. to about 37.5° C. In an embodiment of the present disclosure, a plurality of heating pads are provided in predetermined location of the chamber (111) for heating the chamber (111) and the tissue processing unit (102) when the temperature inside the chamber falls below the predetermined limit. The heating pads are interfaced with the control unit (107), and said control unit (107) regulates the operation of heating pads for maintaining predetermined temperature inside the chamber (111) as required for the tissue digestion process. In another embodiment of the present disclosure, the temperature inside the chamber (111) can be maintained by a method selected from group comprising but not limited to warm air circulation, or use of infra-red heating mechanism or other such technology known in the art.

FIG. 5 is an exemplary embodiment of the present disclosure showing perspective view and magnified view of a filtration assembly (104) of the cell concentration unit (103) of the system (100) for isolating stromal vascular fraction (SVF) cells from adipose tissue. The filtration assembly (104) comprises a plurality of filter chambers (104a-104c) of predetermined shape. And the filtration assistance mechanism/vibratory mechanism is connected to the filtration assembly (104). The filtration assembly (104) is optionally provided with a filter waste chamber (104d) attached to the ultimate filter chamber (104c) for collecting remaining portion of aqueous fraction of tissues after the filtration. In an embodiment of the present disclosure, the shape of the filter chambers (104a-104c) and the filter waste chamber (104d) is selected from group comprising but not limited to cylindrical, rectangular, square, triangular, and trapezoidal shape. Further, each filter chamber is provided with at least one filter cartridge (104e). The filter cartridge (104e) comprises a filter element (A) with predetermined size of perforations disposed in a housing (B). The housing optionally comprises a support member (C). The support member (C) can be selected from the group comprising but not limited to plate with perforations, strainers etc. In an embodiment of the present disclosure, the size of the perforations of the filter element (A) ranges from about 1 μm to about 200 μm.

In an embodiment of the present disclosure, the housing (B) is made hollow and the support member (C) is optionally perforated. The size of the perforation of the support member (C) ranges from about 0.2 mm to 2 mm.

In an embodiment of the present disclosure, the filtration assembly (104) includes filter 1st chamber (104a), input nozzle (104f) for receiving the fluids from the tissue processing unit (102) and a breather nozzle (104g) protected by a breather filter. The filter 1^{st} chamber is provided with a filter cartridge. The filter cartridge comprises of a housing (B), filter element (A) and support member (C). The filter 1^{st} chamber is connected to the filter 2^{nd} chamber (104b). The filter 2^{nd} chamber (104b) consists of a breather nozzle (104g) and a filter cartridge. The filter 2^{nd} chamber (104b) is connected to the filter 3^{rd} chamber (104c). The filter 3^{rd} chamber (104c) consists of a breather nozzle (104g) and a filter cartridge. The filter 3^{rd} chamber (104c) is connected to the filter waste chamber (104d). The filter waste chamber (104d) consists of a waste output nozzle connected to the waste collection unit (106) [shown in FIG. 1]. The waste accumulated in the filter waste chamber (104d) is drained to the waste collection unit (106) and the cells of interest (final product) are collected in the ultimate filter chamber/filter 3^{rd} chamber (104c). The final product from the filter 3^{rd} chamber (104c) is collected into a cell collector through a suitable means. The cell collector includes but is not limited to syringe, cell collection bags or any other cell collection device known in the art.

In an embodiment of the present disclosure, the breather nozzles are protected by a breather filter element of perforations of size ranging from 0.8 μm to 0.1 μm, preferably 0.22 μm, for ensuring aseptic environment. Breather filter element is made of material selected from a group comprising but not limited to PES (polyethersulfone), cellulose acetate, Teflon (PTFE) or any other known in the art.

In an embodiment of the present disclosure, the size of the filter chambers (104a-104c) and the filter waste chamber (104d) is selected based on the requirement. In one of the embodiment, the size of the chambers (104a-104c) will gradually increase in the ascending order (i.e. size of the filter chamber (104c) will be bigger than size of the filter chamber (104b and 104a being the smallest) to carry out effective cell separation.

In an embodiment, the size of the perforations of the first filter element (one between the 1st and 2nd chamber) ranges from about 50-500 μm. In a preferred embodiment, the size of the perforations of the first filter element is 100 μm. Further, the first filter element functions to remove coarse waste such as undigested tissue. In an embodiment, the size of the perforations of the second filter element (one between the 2nd and 3rd chamber) ranges from about 10-50 μm. In a preferred embodiment, the size of the perforations of the second filter element is 30 μm. The second filter element functions to remove fine waste such as undigested tissue and cell aggregates. In an embodiment, the size of perforations of the third filter element (one between the 3rd chamber and filter waste chamber (104d) ranges from about 1-10 μm. In a preferred embodiment, the size of the perforations of the third filter element is 5 μm. The third filter element retains the SVF cell fraction. In a preferred embodiment, the third filter element retains SVF fraction depleted of red blood cells (RBCs). In a more preferred embodiment, the third filter element functions to retain SVF fraction depleted of RBCs, lymphocytes and monocytes, wherein the said RBCs, lymphocytes and monocytes pass through the filter to enter the filter waste chamber (104d).

In an embodiment of the present disclosure, each of the filter chambers (104a-104c) is provided with an output nozzle for collecting the SVF cells. The cells are delivered at gentle pressure and at a particular angle through a set of nozzles which ensures that SVF cells are easily transferred to cell collector like syringe or any other accessories through suitable means for such connection used by the medical attendant for injection or implantation.

In one of the embodiments, the stromal vascular fraction is further enriched in the cell concentration unit (103) by filtering off red blood cells (RBC) on the basis of cell size, by sequential filtration through the filters of different permeability/sizes of the perforation. In another embodiment, red blood cells are depleted using an affinity matrix added during the digestion step, and is removed on the basis of size during sequential filtration through the filters of different sizes of perforations. RBCs bound to the matrix would not pass through the filter and it stays retained in the filter chamber (104a or 104b), and an enriched stromal population comprising the MSC and endothelial progenitor cells would pass through and collect in the ultimate filter chamber (104c). In another embodiment, the RBCs are not removed during filtration.

Further, the filtration assembly (104) can be coupled to a filtration assistance mechanism. The filtration assistance mechanism can be performed by techniques such as but not limiting to simple filtration, pressure assisted filtration, vacuum assisted filtration, vibration assisted filtration or combinations thereof. In a preferred embodiment, the filtration technique followed is vibration assisted filtration.

FIG. 6 is an exemplary embodiment of the present disclosure which illustrates perspective view of the filtration assistance mechanism, the filter vibrator (105) of the system for isolating stromal vascular fraction (SVF) cells from adipose tissue. For effective filtration and to avoid filter element clogging, filter vibrator is employed. The filter vibrator (105) includes a rigid plate (105a) of predetermined shape configured to form a base of the filter vibrator (105). A plurality of guide shafts (105b) fixed at predetermined locations on the rigid plate (105a). Each of the guide shafts (105b) comprises a top stopper at the free end of the guide shaft (105b) and a bottom stopper at predetermined distance below the top stopper. The guide shafts (105b) are arranged to pass through a movable plate (105d) of predetermined shape. The movable plate (105d) is slidably mounted on bottom stopper of the guide shaft (105b). In an embodiment of the present disclosure, the portion of shafts (105b) between the top and bottom stoppers is configured as guide bearing. Further, at least one compression spring (105e) is mounted between the top stopper of the guide shaft (105b) and the movable plate (105d). The compression springs (105d) maintains tension on the movable plate. The filter vibrator further comprises a cam follower (105f) fixed to bottom end of the movable plate (105d) and the cam follower (105f) is configured to follow an amplitude generator (105g). Further, a motor (105h) is mounted on the rigid plate (105a) and the motor (105h) is coupled to the amplitude generator (105g) for actuating the cam follower (105f) to generate vibrations for filtration. In an embodiment of the present disclosure, the shape of the rigid plate (105a) and the movable plate (105d) is selected from a group comprising but not limited to circular shape, square shape, rectangular shape, triangular shape, or any other shape known in the art.

Further, a pair of load bearings (105i) and motor bracket are fixed to the base (105a). The motor (105h) is fixed to the motor bracket and the amplitude generator (105g) is coupled to one of the load bearing (105i). The design of amplitude generator (105g) allows desired amplitude and frequency for effective filtration. The cam follower (105f) is fixed to the movable plate (105d), and rests on the amplitude generator (105g). The compression springs (105e) is provided to ensure that the cam follower (105f) always rests on the amplitude distribution generator (105g) resulting in equal amplitude throughout the process and bringing back the movable plate (105d) to its home position. When the motor (105h) starts running, due to the amplitude generator profile and the cam follower (105f) the impact vibration is achieved, thus resulting in the effective filtration.

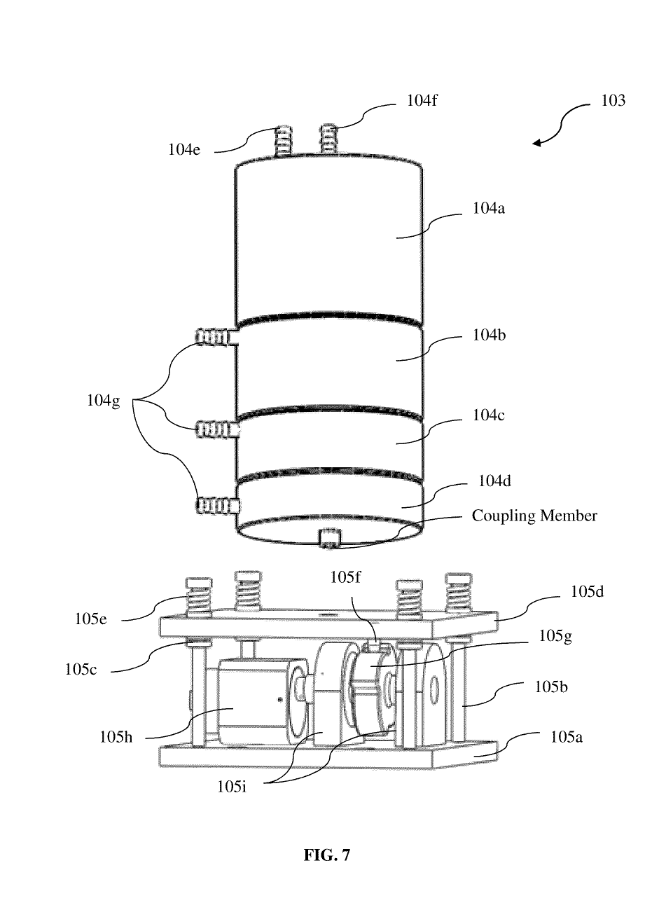

FIG. 7 is an exemplary embodiment of the present disclosure which illustrates perspective view of the cell concentration unit (103) of the system (100) for isolating stromal vascular fraction (SVF) cells from adipose tissue as an embodiment of the present disclosure. The movable plate (105d) of the filter vibrator (105) is connectable to the filtration assembly (104) using the coupling mechanism. The filtration assembly (104) can be connected to the filter vibrator (105) using any method known in the art. In an embodiment of the present disclosure, a threaded hole (105j) is provided in the movable plate (105d) of the filter vibrator (105) and a threaded bolt is provided at bottom surface of the filter waste chamber (104d). The filtration assembly (104) is coupled to the filter vibrator (105) by fastening the threaded bolt into the threaded hole.

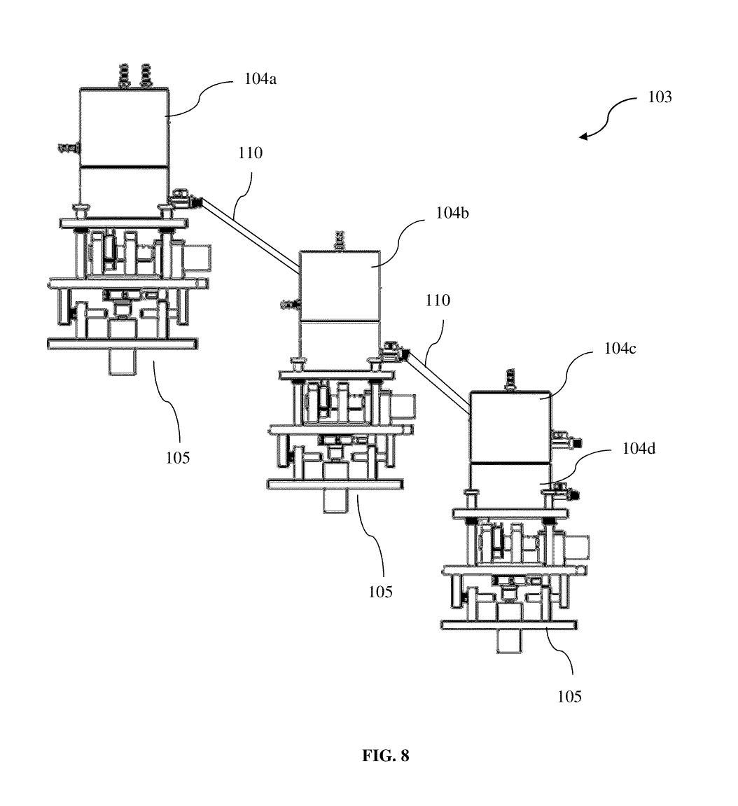

FIG. 8 is an exemplary embodiment illustrating perspective view of the cell concentration unit of the system for isolating stromal vascular fraction (SVF) cells from adipose tissue as another embodiment of the present disclosure. As shown in the FIG. 8, the filter chambers (104a-104c) are arranged/mounted adjacent to each other and the filter waste chamber (104d) is optionally connected to the filter chamber (104c) for collecting the waste tissues or directly connected to the waste unit (106). The filter chambers (104a-104c) are connected to each other using the tubing system (110) for supplying the aqueous fraction of tissue from one chamber to the other. The filter vibrator (105) is positioned below each of the filter chamber (104a-104c) for vibrating the filter chambers (104a-104c) to obtain the SVF cells. Since the filter chambers (104a-104c) are vibrated by the separate filter vibrator (105) the process time is reduced due to differential flow across each filter chamber (104a-104c) based on the size of the filter element. The filter chamber (104c) is provided with an output nozzle/suitable means for collecting the SVF cells. Optionally the filter chambers (104a-104c) are provided with an output nozzle/suitable means for collecting cells. The final SVF cells are delivered at a gentle pressure and at a particular angle through a set of nozzles or suitable means which ensures that SVF cells are easily transferred to a cell collector interfaces like a syringe or any other accessories used by a medical attendant for injection or implantation.

In an embodiment of the present disclosure, each of filter chambers (104a-104c) along with the filter vibrators (105) can be arranged one below the other in descending order to facilitate the flow of tissue between the filter chambers (104a-104c) using gravity. In an alternative embodiment, a motor is provided between each filter chambers (104a-104c) to supply the tissues from one chamber to another chamber.

In an embodiment of the present disclosure, the filter vibrator (105) comprises a horizontal vibrating mechanism comprising a motor coupled to an amplitude generator and a cam follower configured to vibrate the filter chambers (104a-104c) horizontally. In the preferred embodiment, the combination of horizontal vibration and a vertical vibration (best shown in FIG. 8) is used for vibrating the filter chambers (104a-104c).

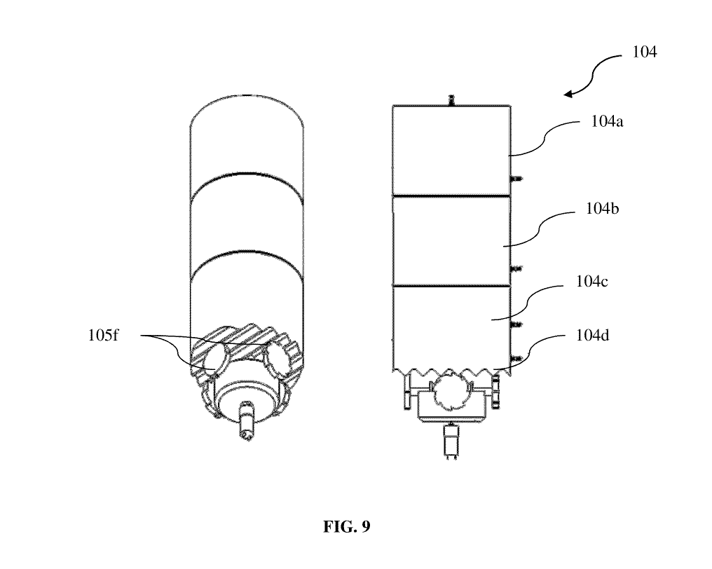

FIG. 9 is an exemplary embodiment of the present disclosure illustrating perspective view and side view of the filtration assembly (104) with plurality of cam followers (105f) mounted in a central axis of the filtration assembly (104) below the filtration unit. As shown in the FIG. 9, plurality of cam followers (105f) are mounted below the filtration assembly (104) and the bottom chamber of the filtration assembly (104) is configured to couple with the cam followers (105f) to create localized vibration along the circumference of the filtration assembly (104) to increase the efficiency of SVF processing by increasing the flow rate. In an embodiment of the present disclosure, the bottom filter chamber and the cam followers (105f) are configured as worm drive. Thus the localized vibration is generated along the circumference of the filtration assembly (104) when the multiple cam followers (105f) are rapidly rotated using the motors.

In a preferred embodiment of the present disclosure, the stromal vascular fraction is obtained by following the process steps as mentioned below—
a. a predetermined quantity of a tissue sample and a wash buffer solution contained in containers (101a and 101b) is supplied to a tissue processing unit (102);
b. tissue samples are washed with wash buffer solution by agitating the mixture in the tissue processing unit (102); the wash step is repeated for about 1-6 times preferably 3-4 times.
c. the mixture is separated in to primary fatty upper fraction and a primary aqueous lower fraction in the tissue processing unit (102) by allowing phase separation of the mixture;
d. the primary lower aqueous fraction obtained in previous step is disposed to a waste collection unit (106);
e. a predetermined quantity of a digestive buffer contained in an digestive buffer container (101c) is supplied to the tissue processing unit (102);
f. the fatty upper fraction is mixed with the digestive buffer by agitating the mixture in the tissue processing unit (102) for a predetermined time to carry out the digestion process, and optionally the digestion process is arrested at the end of the predetermined time period, by pumping in a predetermined quantity of serum or enzyme inhibitor or a combination thereof, mixing by agitation;
g. the mixture is separated in to a secondary fatty upper fraction and a secondary aqueous lower fraction by allowing phase separation of the mixture in the tissue processing unit (102);
h. the secondary aqueous lower fraction obtained in previous step is directed to a cell concentration unit (103); and
i. filtering the secondary aqueous fraction within the cell concentration unit (103), comprising of filtration assembly (104), optionally along with removal of red blood cells to obtain said SVF cells.

In an embodiment of the present disclosure, the wash buffer is selected from group comprising normal saline, ringer's solution, lactated ringer's solution, hanks' balanced salt solution and any combination thereof. The washing process comprises of at least one wash step.—In a preferred embodiment, the washing process comprises of three-four wash steps, and the complete washing process is carried out for time period ranging from about 5 minutes to about 20 minutes, preferably about 10 minutes.

In an embodiment of the present disclosure, the digestion process is carried out for time period ranging from about 15 minutes to about 2 hours, preferably from about 30 minutes to about one hour.

In an embodiment of the present disclosure, the digestive buffer is a mixture of wash buffer and digestive buffer, wherein the digestive buffer is selected from a group not limited to comprising collagenase, pepsin, trypin and dispase or any combination thereof.

In an embodiment of the present disclosure, at the end of the digestion process, a pre-calculated volume ranging from about 1 ml to about 300 ml, preferably about 10 ml to 100 ml of—serum is added optionally to inactivate the enzyme of digestive buffer solution. In another embodiment, an enzyme inhibitor, not limited to EGTA, cysteine, or N-acetyl cysteine or similar chemically-defined inhibitor is added to inactivate the enzyme. In yet another embodiment, the enzyme is not inactivated as the extensive washing of the digested cells is sufficient to completely remove the enzyme.

In an embodiment of the present disclosure, the phase separation occurs in time period ranging from about 1 minute to about 10 minutes, preferably from about 2 minutes to about 5 minutes.

In an embodiment of the present disclosure, washing with the wash buffer and the digestive buffer is carried at temperature ranging from about 35° C. to about 38° C. preferably from about 36.5° C. to about 37.5° C.

In an embodiment of the present disclosure, the optional removal of red blood cells is carried out by at least one of filtration or affinity matrix or a combination thereof.

In an embodiment of the present disclosure the system (100) can be used to isolate the cells from the mammalian tissue selected from a group comprising but not limited to adipose tissue, placental tissue and umbilical cord tissue.

The composition of the tissue sample at various stages of processing are as follows:
Initial tissue sample before processing comprises the intact adipose tissue with blood and tumescent fluids such as saline, lidocaine and epinephrine.
After the wash process and phase separation, the retained primary fatty fraction comprises intact adipose tissue free from blood and tumescent fluids such as saline, lidocaine and epinephrine.
After digestion process and before phase separation, the composition comprises of dissociated adipose tissue with fatty and aqueous phases.
After phase separation, the partitioned secondary aqueous fraction contains SVF cells, along with undigested tissue waste and blood cells such as RBC, lymphocytes and monocytes.
After filtration, the final composition (SVF fraction) comprises mesenchymal stem cells, endothelial progenitor cells, mature endothelial cells, and a limited population of immune cells, RBC and preadipocytes, and limited population of fibroblasts and smooth muscle cells.

Except for the modules used during the process, all other mechanical and electronic parts of the device are housed in the chamber (111) away from the sample pathway to prevent accidental spillage of samples and contamination of moving parts of the device. Further, no mechanical parts of the device are exposed or in contact with the sample during the process. All tubing systems (110) and containers (101a-101c) used for processing are disposable and intended for one-time use. Because of this design, one can use this device in a clinical setting without any risk of cross-contamination of tissue samples. The display screen displays each step of the process and records various parameters of the process during operation in real-time for later reference and audit.

The present disclosure is further elaborated with the help of following examples and associated figures. However, these examples should not be construed to limit the scope of the present disclosure.

### EXAMPLES

### Example 1

### Processing of SVF

The use of stromal vascular stem cells from adipose tissue obtained from lipoaspirated fat tissue has important implications in autologous transplantation for various cosmetic applications. SVF is a heterogeneous cell mixture comprising of preadipocytes, mature endothelial cells (EC), endothelial progenitor cells (EPC), vascular smooth muscle cells (SMC), pericytes, mural cells, macrophages, fibroblasts, mesenchymal stem cells (MSC) and their progenitors. Good quality cells with high viability are obtained by recovering the digested cells by a process of repeated phase separation and sequential filtration.

### Advantages of the Present Invention

The system (100) disclosed in the present disclosure describes a compact bench-top system for point-of-care isolation of SVF cells from adipose tissue. The system (100) comprises of a durable framework chamber (111) housing the electrical and electronic components, pumps (108) etc. It further includes a closed, sterile, disposable flow path for tissue processing comprising of the tissue processing unit (102) and cell concentration unit (103), containers (101a-101c), tubing systems (110) and connectors. The system (100) uses an optimized process for isolation of SVF cells from adipose tissue without employing the technique of centrifugation. Elimination of the bulky centrifuge results in a compact system with a small footprint that can be easily accommodated in a clinical setting. The cells recovered by this process are also not subjected to the stress of centrifugal forces. The present disclosure is economical owing to its simplicity and the nature of the materials used; and is easy to operate and has the flexibility to accept fat tissue from most commonly used lipoaspirators. A cleverly designed geometry of the tissue processing unit (102) and the cell concentration unit (103) ensures gentle cell isolation with maximal efficiency and cell viability. It also provides a xeno-free isolation process where no animal derived products are used. The filtration assembly (104) produces a final cell product that is enriched for cells of therapeutic benefit comprising of MSC and their progenitors, EPC, EC, preadipocytes, smooth muscle cells etc., and free from contaminating cells such as RBC.

The modular nature of the disposables provides the clinic with the flexibility of using units of different capacity to process small or large volumes of fat tissue.

Further advantage of using the automated system (100) of present disclosure for processing SVF includes lack of contamination of the final product with red blood cells. It is an elegant, simple and novel way to isolate stromal vascular fraction cells (SVF) from adipose tissue harvested through liposuction. Since the whole process operates in a closed, sterile environment, the isolated cells from this system can be used for autologous transplantation in patients. Secondly, the system (100) enables gentle handling of cells throughout the process to concentrate cells. Thirdly, the modularity of the system (100) along with one-time use accessories prevents cross-contamination of samples and enhances safety of the product intended.

The process, in its desired form, comprises of the following steps:
Transfer of tissue from surgical container to the system by means of a pump.
Washing the tissues repeatedly through agitation, mixing and phase separation.
Digesting the tissue with—digestive buffer to release associated cells from fat tissue.
Recovering the cells thus released in the aqueous medium by phase separation.
Concentrating the cells in a workable volume by a series of filtration steps.

The cells isolated by this method comprises of mesenchymal stem cells, endothelial progenitor cells, mature endothelial cells, and a limited population of immune cells and preadipocytes, all of which have been shown to be present in SVF obtained from lipoaspirated tissue.

### Example 2

### Operation of the Automated System for Processing the SVF

FIG. 4 illustrates the sequential process steps of SVF processing from the adipose tissue. The tissue samples obtained from surgery is transferred into the tissue container (101a) of the system (100). To provide maximum flexibility to surgeons using various commercially available liposuction instruments, the inlet tubing system into tissue processing unit is designed to accept various liposuction containers currently in use for such surgical procedures. The tissue in the tissue container (101a) is pumped into the tissue processing unit (102) by means of a peristaltic pump (108) which ensures controlled flow, via a 5 way manifold through an inlet pinch valve. Buffer solution in the buffer container (101b) is pumped into tissue processing unit (102) by means of a peristaltic pump (108) which ensures controlled flow, via a 5 way manifold through an inlet pinch valve. The wash process in the tissue processing unit (102) is carried out by operating the agitation mechanism. After completion of the wash process, phase separation is carried out for a specified time which is variable. The waste fraction is collected after the phase separation in the bottom half of the tissue processing unit (102) and is pumped into the waste collection chamber (106) through an outlet via an outlet pinch valve, with controlled flow by means of a peristaltic pump (108). In an embodiment, the above wash process is carried out for 3 times which is variable. The wash process is followed by the digestion process wherein, a specified quantity of digestive buffer from the digestive buffer container (101c) is pumped into the tissue processing unit (102). The time period for said digestion process is variable. Optionally the digestion process is arrested at the end of the predetermined time period, by pumping in a predetermined quantity of serum or enzyme inhibitor or a combination thereof, by agitation/mixing. An aqueous fraction is obtained after the phase separation, which is pumped into the cell concentration unit (103) through a peristaltic pump (108) via an outlet pinch valve. Filtration is carried out and after the filtration process, the concentrated cells are aspirated and collected in the cell collector such as syringe. The collected cells can be directly injected into the patient for autologous transplantation or can be used for further culturing for growth or differentiation of the cells. In an embodiment, during the wash and digestion process, the ambient temperature and the temperature of tissue-digestive buffer mixture is maintained at 37°±0.5° C. and the said temperature is controlled by the heater and a temperature sensor (113) [shown in FIG. 3].

The peristaltic pumps (108), the filtration assembly (104), tissue processing unit (102), heating pads and the temperature sensor (113) are interfaced to a controller (107) [as shown in FIG. 3]. The controller (107) is programmed to carry out the process of isolating SVF cells automatically.

### Example 3

### Comparative Study of Various Cell Separation Techniques

### Effect of Repeated Centrifugation on SVF Yield.

The below figure demonstrates progressive loss of SVF cells with every centrifugation step, in the manual process of SVF isolation.

TABLE 1
Comparison of SVF isolation by centrifugation vs. filtration techniques.
Filtration yields a higher recovery of SVF cells as compared to the
manual process.

| | Conventional Process | Device process |
|---|---|---|
| Sample | (Centrifugation) | (Filtration) |
| Sample 1 | 100% | 115% |
| Sample 2 | 100% | 117% |
| Sample 3 | 100% | 103% |

TABLE 2
Comparison of SVF viability by centrifugation vs. filtration techniques.
Viability of SVF isolated by centrifugation and filtration found to be
comparable, and >97%

| | Conventional Process | Device process |
|---|---|---|
| | (Centrifugation) | (Filtration) |
| Viability (n = 3) | 97.3 ± 1.5% | 97.5 ± 2.8% |

TABLE 3
Comparison of SVF composition, obtained by centrifugation vs. filtration
techniques. Table represents mean percentage positive cells with standard
error, from five different data sets. Data shows evidence of reduction in
RBC contamination, and enrichment of ASC and EPC cell populations in
the SVF obtained by filtration, as compared to centrifugation. ASC =
adipose derived stem/stromal cells; EPC = endothelial progenitor cells;
RBC = red blood cells.

| Cell Type | Conventional Process | Device process |
|---|---|---|
| (Marker Profile) | (Centrifugation) | (Filtration) |
| ASC | 22.8 ± 2.5% | 30.3 ± 5.9% |
| (CD34+ CD90+ CD105+ | | |
| CD31−) | | |
| EPC | 20.6 ± 10.5% | 26.7 ± 5.4% |
| (CD34+ CD31+) | | |
| RBC | 20.1 ± 15% | 10.7 ± 10% |
| (Glycophorin A+) | | |

### EQUIVALENTS

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims

### REFERRAL NUMERALS

| Reference Number | Description |
|---|---|
| 100 | SVF processing system |
| 101a | Buffer solution container |
| 101b | Tissue container |
| 101c | Digestive buffer container |
| 102 | Tissue processing unit |
| 103 | Cell concentration unit |
| 104 | Filtration unit/assembly |
| 104a-104c | Filter chamber |
| 104d | Filter waste chamber |
| 104e | Filter cartridge |
| A | Filter element |
| B | Filter housing |
| C | Support member |
| 104f | Fluid inlet |
| 104g | Breather nozzle |
| 105 | Filter vibrator |
| 105a | Base of the Filter vibrator |
| 105b | Guide shafts of the Filter vibrator |
| 105c | Guide bearing of the Filter vibrator |
| 105d | Movable plate of the Filter vibrator |
| 105e | Compression spring of the Filter |
| | vibrator |
| 105g | Amplitude generator of the Filter |
| | vibrator |
| 105f | Cam follower of the Filter vibrator |
| 105h | Motor of the Filter vibrator |
| 105i | Load bearing of the Filter vibrator |
| 105j | Threaded hole on movable plate |
| 106 | Waste collection chamber |
| 107 | Control unit |
| 108 | Peristaltic pump |
| 109 | Valves |
| 110 | Tubing system |
| 111 | Chamber |
| 112 | Display unit |
| 113 | Temperature sensor |

1. A system (100) for isolating cells by processing of tissue, said system (100) comprising:
plurality of containers (101a-101c) each for storing at least one of digestive and wash buffer solutions, tissue samples and digestive buffer;
a tissue processing unit (102) fluidly connectable with the containers (101a-101c) for receiving the digestive buffer, tissue samples and wash buffer solutions, and processing the tissue samples, wherein the tissue processing unit (102) performs at least one of the washing processes, digestion process, phase separation process and combination thereof for separating an aqueous fraction and a fatty fraction from the digested tissue samples;
a cell concentration unit (103) fluidly connectable with the tissue processing unit (102) for filtering the aqueous fraction of the digested tissue, wherein the concentration unit (103) comprises:
a filtration assembly (104) including:
plurality of filter chambers (104a-104c) for sieving the aqueous fraction of digested tissues to collect cells;
a filter vibrator (105) placed below the filter chambers (104a-104c) for vibrating the filter chambers (104a-104c);
a waste collection unit (106) fluidly connectable to the tissue processing unit (102) and the filtration assembly (104), for receiving at least one of aqueous fraction of tissues and fatty fraction of tissues from the tissue processing unit (102) and the filtration assembly (104); and
a control unit (107) interfaced with the tissue processing unit (102), and the filter vibrator (105) for controlling the operation of the tissue processing unit (102) and the filter vibrator (105) to obtain the cells from the tissue.

2. The system as claimed in claim 1, wherein the tissues are mammalian tissues selected from at least one of adipose tissue, placental tissue and umbilical cord tissue.

3. The system as claimed in claim 2 isolates Stromal Vascular Fraction (SVF) cells by processing the adipose tissue, and multipotent stem/stromal cells from placental and umbilical cord tissue.

4. The system (100) as claimed in claim 1 comprises plurality of peristaltic pumps (108) and valves (109) connectable to the containers (101a-101c), tissue processing unit (102), cell concentration unit (103) and waste collection unit (106) for controlling flow rate of tissue sample, the buffer solution, the digestive buffer solution and a waste fluids.

5. The system (100) as claimed in claim 1, wherein the containers (101a-101c), the tissue processing unit (102), the cell concentration unit (103), and the waste collection unit (106) are connected to each other through tubing system (110).

6. The system (100) as claimed in claim 1, wherein the system (100) is optionally enclosed in a chamber (111).

7. The system (100) as claimed in claim 1, wherein the control unit (107) is provided with a user interface having a display unit (112) and input buttons to feed in required parameters for processing the tissue.

8. The system (100) as claimed in claim 1 comprises at least one temperature sensor (113), placed in a chamber (111) to measure and regulate the temperature of the chamber (111), wherein the temperature sensor (113) is interfaced with the control unit (107) to maintain the temperature of the chamber (111) within a predetermined limit.

9. The system (100) as claimed in claim 1 comprises at least one input nozzle (104f) provided in first chamber of the filtration assembly (104) for receiving digested aqueous fraction of tissues from the tissue processing unit (102).

10. The system as claimed in claim 1 comprises at least one breather nozzle (104g) provided in each of the filter chambers (104a-104c) to facilitate free air flow inside the chambers (104a-104c).

11. The system (100) as claimed in claim 1 comprises at least one filter cartridge (104e) located in—each of the filter chambers (104a-104c).

12. The system (100) as claimed in claim 11, wherein the filter cartridge (104e) comprises a filter element (A) having predetermined perforations disposed in a housing (B), wherein the housing (B) optionally comprises a support member (C) at its bottom.

13. The system (1) as claimed in claim 11, wherein size of the perforations of the filter element (A) is ranging from about 1 μm to about 200 μm.

14. The cell concentration unit (103), comprising:
a filtration assembly (104) including:
plurality of filter chambers (104a-104c) of predetermined shape and predetermined size; and
at least one filter cartridge (104e) located in each of the filter chambers (104a-104c), wherein the filter cartridge (104e) comprises a filter element (A) having predetermined perforations disposed in a housing (B), wherein the housing (B) optionally comprises a support member (C) at its bottom;
a filter vibrator (105) placed below the filter chambers (104a-104c) for generating vibrations for filtration.

15. The unit as claimed in claim 14 comprises at least one input nozzle (104f) provided in the first chamber (104a) of the filtration assembly (104) for receiving fluid for filtration.

16. The unit as claimed in claim 14 comprises at least one breather nozzle (104g) provided in each of the filter chambers (104a-104c) to facilitate free air flow inside the chambers (104a-104c).

17. The unit as claimed in claim 14 optionally comprises a filter waste chamber (104d) connected to at least one of the filter chambers (104a-104c).

18. The unit as claimed in claim 14, wherein the filter chambers (104a-104c) and the filter waste chamber (104d) are mounted one above the other.

19. The unit as claimed in claim 14, wherein the filter chambers (104a-104c) and the filter waste chamber (104d) are mounted adjacent to each other.

20. The unit as claimed in claim 19, wherein each of the filter chambers (104a-104c) and the filter waste chamber (104d) are connected using a tubing system (110).

21. The unit as claimed in claim 14, wherein the filter vibrator (105) comprises:
a rigid plate (105a) of predetermined shape configured to form a base of the filter vibrator (105);
a plurality of guide shafts (105b) fixed at predetermined locations on the rigid plate (105a), wherein each of the guide shaft (105b) comprises a top stopper at the free end of the guide shaft (105b) and a bottom stopper at predetermined distance below the top stopper, wherein the guide shafts (105b) are arranged to pass through a movable plate (105d);
the movable plate (105d) of predetermined shape is slidably mounted on bottom stopper of the guide shaft (105b), wherein the movable plate (105d) is connectable to the filtration assembly (104);
at least one compression spring (105e) mounted between the top stopper of the guide shaft (105b) and the movable plate (105d);
at least one cam follower (105f) fixed to bottom end of the movable plate (105d), wherein the cam follower (105f) is configured to follow an amplitude generator (105g); and
at least one motor (105h) mounted on rigid plate (105a), wherein the motor (105h) is coupled to the amplitude generator (105g) for actuating the cam follower (105f) to generate vibrations for filtration.

22. The unit as claimed in claim 21 comprises a pair of load bearings (105i) mounted on rigid plate (105a), and are coupled to the amplitude generator (105g).

23. The unit as claimed in claim 21, wherein a portion between the top and bottom stoppers of each guide shaft (105b) is configured as guide bearing element (105c).

24. A method of obtaining Stromal vascular fraction (SVF) cells from adipose tissue using the system (100) as claimed in claim 1, said method comprising acts of:
a. receiving predetermined quantity of a tissue sample and a wash buffer solution contained in a containers (101a and 101b) by a tissue processing unit (102);
b. washing the tissue samples with wash buffer solution by agitating the mixture in the tissue processing unit (102);
c. allowing phase separation of the mixture to obtain a primary fatty upper fraction and a primary aqueous lower fraction in the tissue processing unit (102);
d. disposing the primary lower aqueous fraction obtained in step (c) to a waste collection unit (106);
e. pumping predetermined quantity of a digestive buffer contained in a digestive buffer container (101c) to the tissue processing unit (102);
f. digesting the fatty upper fraction with the digestive buffer by agitating the mixture in the tissue processing unit (102) for a predetermined time;
g. Optionally arresting the digestion process at the end of the predetermined time period, by pumping in predetermined quantity of serum or enzyme inhibitor or a combination thereof, mixing by agitation
h. allowing phase separation of the mixture in the tissue processing unit (102) to obtain a secondary fatty upper fraction and a secondary aqueous lower fraction;
i. directing the secondary aqueous lower fraction to a cell concentration unit (103); and
j. filtering the secondary aqueous fraction within the cell concentration unit (103) by vibrating a filtration assembly (104) of the cell concentration unit (103) by a filter vibrator (105), optionally along with removal of red blood cells to obtain said SVF cells.

25. The method as claimed in claim 24, wherein the steps (b-d) are performed at least one time, preferably 3-4 times.

26. The method as claimed in claim 25, wherein the step (b-d) is carried out for time period ranging from about 5 minutes to about 20 minutes, preferably about 10 minutes.

27. The method as claimed in claim 24, wherein the step (f) is carried out for time period ranging from about 15 minutes to about 2 hours, preferably from about 30 minutes to about one hour.

28. The method as claimed in claim 24, wherein the phase separation occurs in time period ranging from about 15 Seconds to about 10 minutes, preferably from about 2 minutes to about 5 minutes.

29. The method as claimed in claim 24, wherein the digestive buffer is a mixture of wash buffer and digestive buffers, wherein the digestive buffers is selected from group comprising collagenase, pepsin, trypin and dispase or any combination thereof.

30. The method as claimed in claim 24, wherein the wash buffer is selected from group comprising normal saline, ringer's solution, Hank's balanced salt solution (HBSS) lactated ringer's solution and any combination thereof.

31. The method as claimed in claim 24, wherein the second aqueous fraction of step (h) comprises mixture of SVF cells, undigested tissue waste, RBC, lymphocytes and monocytes or any combination thereof.

32. The method as claimed in claim 24, wherein washing with the wash buffer and the digestive buffer is carried at temperature ranging from about 35° C. to about 38° C. preferably from about 36.5° C. to about 37.5° C.

33. The method as claimed in claim 24, wherein the optional removal of red blood cells is carried out by at least one of filtration or affinity matrix or a combination thereof.

34. The system and the method as claimed in claim 1, wherein the obtaining of the SVF is automated and maintains sterility throughout the process.

35. The system and the method as claimed in claim 24, wherein the obtaining of the SVF is automated and maintains sterility throughout the process.
